(19) United States
(12) Patent Application Publication
(10) Pub. No.: US 2009/0155352 A1
(43) Pub. Date: Jun. 18, 2009

(54) Microemulsion containing indolocarbazole compound and dosage forms containing the same

(75) Inventors: Anthony S. Drager, Thorndale, PA (US); Bradley T. Mclntyre, Thorndale, PA (US); Piyush R. Patel, Wallingford, PA (US)

Correspondence Address: Ross J. Oehler;CEPHALON, Inc., 41 MOORES ROAD, PO BOX 4011, FRAZER, PA, 19355, US

(73) Assignee: Cephalon, Inc., Frazer, PA (US)

(21) Appl. No.: 12/313,476

(22) Filed: Nov. 20, 2008

Related U.S. Application Data
(60) Provisional application No. 61/003,780, filed on Nov. 20, 2007.

(51) Int. Cl. A61K 9/48; A61K 31/553

(52) U.S. Cl. 424/451; 514/211.08

(57) ABSTRACT

The invention described herein provides a pharmaceutical composition and oral dosage forms containing the same, having high concentrations of solubilized indolocarbazole compounds as the active ingredient in microemulsion form. The invention also provides a process for increasing the solubilized concentration of indolocarbazole compounds such as lestaurtinib using the addition of water in combination with a hydrophilic component as part of the microemulsion formation process.

The present invention relates to pharmaceutical compositions for oral dosage forms. In particular, the invention pertains to pharmaceutical compositions in microemulsion form having high concentrations of solubilized indolocarbazole compounds as the active ingredient.

### BACKGROUND OF THE INVENTION

The indolocarbazole alkaloid compound [9S-(9α,10β,12α)]-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one (CAS Registry Number 111358-88-4) is an orally bioavailable receptor-tyrosine kinase inhibitor that can be prepared as a chemically synthesized derivative of K-252a, which is a fermentation product of Nonomurea longicatena. This compound is described in U.S. Pat. No. 4,923,986—the entire text of which is incorporated herein by reference. The indolocarbazole compound [9S-(9α,10β,12α)]-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one, also known under the generic name lestaurtinib, has the following structural formula:

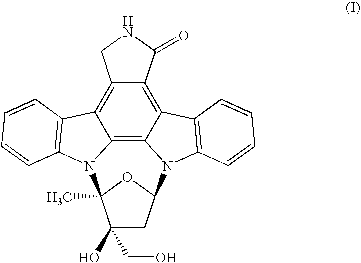

Compositions comprising Formula (I), or lestaurtinib, can include aqueous solutions as described in U.S. Pat. No. 5,599,808. Particle-forming compositions and microemulsion pre-concentrate solutions of lestaurtinib, and related indolocarbazole compounds, are described in U.S. Pat. No. 6,200,968 (the '968 patent), for example. Solid state solutions of Formula (I) are disclosed in U.S. Publ. Application No. 2002/0020176 (the '176 publication). The text of these references is incorporated herein by reference.

Various therapeutic treatments have been associated with indolocarbazole alkaloid compounds such as lestaurtinib. For instance, U.S. Pat. No. 5,765,494 describes use of lestaurtinib and related indolocarbazole compounds for the treatment of neurological disorders such as Alzheimer's disease, motor neuron disease, amyotrophic lateral sclerosis (ALS), Parkinson's disease, cerebrovascular disease, cerebrovascular conditions, such as ischemia, AIDS dementia, epilepsy, concussive injuries to brain, spinal cord, penetrating injuries to brain, spinal cord, and Huntington's disease.

U.S. Pat. No. 5,516,772 describes lestaurtinib and related compounds as being useful in enhancing neurotrophin-induced activities of neurotrophin responsive cells (e.g., cholinergic, sensory or DRG neurons), which is a feature of many human neurological disorders, including, but not limited to, Alzheimer's disease; motor neuron disorders (e.g., ALS, Parkinson's); cerebrovascular disorders (e.g., stroke, ischemia); Huntington's disease; AIDS dementia; epilepsy; concussive or penetrating injuries of the brain or spinal cord; peripheral neuropathies (e.g., those affecting DRG in chemotherapy-associated peripheral neuropathy); and disorders induced by excitatory amino acids.

U.S. Pat. No. 5,654,427 describes use of lestaurtinib and related compounds for the treatment of pathological conditions of the prostate, such as benign prostatic hypertrophy, or prostatic cancer, i.e., locally confined or metastatic prostate cancer. U.S. Pat. No. 5,985,877 describes its use in combination with chemical castration agents, such as estrogens; LHRH agonists, e.g., leuprolide acetate and goserelin acetate; LHRH antagonists, e.g., ANTIDE® (Ares-Serono) and GANIRELIX® (Akzo Nobel); and antiandrogens, e.g., flutamide and nilutamide for the treatment of prostate cancer.

Formula (I), or lestaurtinib, is also described in U.S. Pat. No. 6,448,283 as being useful for the prevention and treatment of hearing loss and loss of the sense of balance, and in particular for preserving sensory hair cells and cochlear neurons in a subject. PCT Publ. No. 02/080937 describes its use in combination with an antineoplastic agents such as fluoropyrimidines, including 5-fluorouracil and ftorafur; pyrimidine nucleosides, such as gemcitabine, 5-azacytidine; and cytosine arabinoside and purines, such as 6-thioguanine for the treatment of patients with cancer. U.S. patent application Ser. No. 11/222,409, filed Sep. 8, 2005, now published Patent Application No. 2006/0058250 published Mar. 16, 2006, describes use of Formula (I) and related compounds for the treatment of proliferative skin disorders including various forms of psoriasis, such as psoriasis vulgaris and psoriasis eosinophilia.

One problem associated with indolocarbazole compounds such as lestaurtinib has been preparing solid formulations with these compounds in solubilized form. More specifically, the indolocarbazole compound of Formula (I), lestaurtinib, has been problematic to formulate into pharmaceutical compositions due to its large macrocyclic ring structure and lack of peripheral alkyl substitution. For instance, it had been found to have a poor water solubility (1.7 μg/mL at 22° C.), and thus poor bioavailability. Some methods of formulating lestaurtinib include use of microemulsion pre-concentrate, as disclosed in the '968 patent and solid state solutions, as disclosed in the '176 publication. However the maximum solubilization of lestaurtinib in the solid state solutions has been found to be about 3% by weight, or at a concentration of 29 mg/g. Thus, previous efforts to solubilize lestaurtinib have resulted in relatively large compositional volumes in order to achieve a pharmaceutically appropriate dosage regimen. Consequently, there is ongoing interest in the pharmaceutical field for accomplishing successful solubilized and stable formulations and dosage forms containing the above indolocarbazole compound for therapeutic administration.

Preliminary clinical data for lestaurtinib suggests that a twice daily dose level of 80 mg of the active ingredient would be required to reach the desired efficacy in patients. The existing capsule formulation allowed for a maximum solubility of 29 mg/ml of the active ingredient, only enabling the preparation of a 20 mg capsule dosage form which would require 8 capsules per day to achieve the daily dosage. This formulation was a self-emulsifying drug delivery system (SEDDS) comprised of a blend of polyoxyethylene glycols (PEGs) and MYRJ® 52 (polyoxyethylene 40 stearate surfactant available from Croda N.A., Parsippany, N.J.). Difficulties also existed with manufacturability of this dosage form as a result of the fill volumes required to obtain the 20 mg dosage form and available apparatus capabilities. In summary, difficulties have been encountered in efforts to formulate concentrated, stable, and convenient dosage forms containing highly insoluble indolocarbazoles.

There exists a need in the pharmaceutical field for improved pharmaceutical compositions containing highly insoluble indolocarbazole compounds, such as lestaurtinib. There is a further need for pharmaceutical compositions that are stable formulations containing increased or high amounts of solubilized concentrations of indolocarbazoles as compared to previous dosage forms, and for pharmaceutical compositions containing indolocarbazoles which are convenient to administer. There is further need for improved formulations that achieve high concentrations of indolocarbazoles such as lestaurtinib while preserving bioavailability of the active ingredient.

### SUMMARY OF THE INVENTION

The invention provides an improved microemulsion-based formulation comprising relatively high solubilized concentrations of highly insoluble pharmaceutically active compounds known as indolocarbazoles. It has been discovered that during the preparation of microemulsions having indolocarbazole compounds as the active ingredient, the addition of water during the admixture steps substantially increases the obtainable amount of solubilized indolocarbazole. For instance, it has been discovered that solubilized concentrations of up to about 9% by weight of the total composition can be achieved, or concentrations of the active ingredient at greater than 85 mg/g. This is in contrast to the expected phenomenon typically associated with the chemical nature of highly water-insoluble indolocarbazole compounds and previous formulation efforts. Furthermore, the desired bioavailability criteria of the active ingredient is nevertheless achieved. The invention is particularly useful in formulating the indolocarbazole alkaloid compound, [9S-(9α,10β,12α)]-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one, which goes by the geneic name lestaurtinib.

Put another way, the invention provides a solid composition suitable for (gelatin) encapsulation wherein relatively high concentrations of solubilized active indolocarbazole ingredient can be achieved for a given total fill volume, thereby permitting smaller fill volumes to be utilized and therefore smaller capsule sizes and/or administration of comparatively fewer capsules to accomplish the same or similar desired bioavailability of the active ingredient as compared to lower concentration regimens. Thus, the invention permits either a decreased fill volume, i.e., smaller capsule for a given dosage, or higher capsule potency by maintaining the same volume capsule and increasing the dosage of active within. Thus, greater patient comfort, convenience and lower manufacturing costs can be achieved for lestaurtinib, for example, over previous capsular dosage forms.

The pharmaceutical composition of the present invention can exhibit a Cₘₐₓ that is at least 1.5 to two times greater, and even about 3 times greater, than the Cₘₐₓ that is observed with conventional indolocarbazole microemulsion pre-concentrate formulations, and a shorter Tₘₐₓ than that which is observed with conventional indolocarbazole microemulsion formulations.

In one aspect, the invention provides a pharmaceutical composition comprising: an indolocarbazole compound present in a solubilized concentration ranging from at least about 3% up to about 9% by weight of the total composition; a hydrophilic polymer component; and water; wherein the composition is a microemulsion. In one embodiment, the indolocarbazole compound is lestaurtinib. In a preferred embodiment, the composition further comprises a surfactant and antioxidant component.

In a further aspect of the invention, the invention provides a capsular dosage form comprising a pharmaceutical fill composition, the composition comprising: indolocarbazole compound present in an amount ranging from about 3 to about 9% per total composition weight; a hydrophilic polymer component present in an amount ranging from about 30% to about 95% per total composition weight; and water present in an amount ranging from about 0.8% to about 50% by weight of total composition weight; wherein the composition is a microemulsion formulated for encapsulation with a hard capsule material.

The invention also provides a pharmaceutical composition comprising a microemulsion having an indolocarbazole compound as an active ingredient present in a solubilized concentration of at least about 3% of the total weight, said composition being prepared by: combining an indolocarbazole compound and hydrophilic polymer ingredient; adding water and mixing at a temperature from about 40° C. to about 100° C.; and forming a microemulsion, wherein the indolocarbazole is present in a concentration of at least about 30 mg/g.

In another aspect, the invention provides a process for increasing the solubilized concentration of an indolocarbazole compound in a microemulsion for a given fill volume, said process comprising: adding water to a molten mixture of indolocarbazole compound, and hydrophilic polymer ingredient; mixing at a temperature from about 40° C. to about 100° C.; and forming a microemulsion; wherein the solubilized concentration of indolocarbazole present in the resulting microemulsion is at least 3% and can be up to about 9% total composition weight.

In yet another aspect, the invention provides a method of inhibiting receptor-tyrosine kinase in a recipient comprising orally administering to a recipient in need of such treatment an oral dosage form having a pharmaceutical composition comprising: an indolocarbazole compound present in a solubilized concentration of at least about 3% by weight of the total composition; a hydrophilic polymer component; and water; wherein the composition is a microemulsion.

These and other advantages associated with the invention will become apparent from the following detailed description.

### BRIEF DESCRIPTION OF THE DRAWINGS

The following drawings further illustrate the invention and are not to be construed as imparting necessary limitations on the invention:

FIG. 1 is a graph plotting percent water versus mg/g drug load and entitled “Water Required for Microemulsion Formation”, according to one embodiment of the invention.

FIG. 2 is a pair of photographs showing the appearance of formulations before and after the addition of water according to one embodiment of the invention.

FIG. 3 is a graph showing the relationship between particle size upon dilution of the formulation (nm) and varying % surfactant (MYRJ® 52) content according to one embodiment of the invention.

FIGS. 4A and 4B together show dissolution profiles as affected by varying PEG molecular weights in the formulation according to one embodiment of the invention. FIG. 4A is the dissolution profile using 95% PEG/5% MYRJ® 52 vehicle formulation. FIG. 4B is the dissolution profile using 50% PEG/50% MYRJ®52 vehicle formulation.

FIG. 5 is a graph showing comparative plasma levels (serum concentrations in ng/ml) for a 40 mg/kg dose of lestaurtinib formulations of different concentrations versus time (hrs) from in vivo testing in rats using the liquid formulation as a control.

FIG. 6 is a graph showing comparative plasma levels (serum concentrations in ng/ml in rat model) of lestaurtinib versus time (hrs) using varying % surfactant amounts.

FIG. 7 is a graph showing comparative plasma levels (serum concentrations in ng/ml in rat model) of lestaurtinib versus time (hrs) using varying PEG molecular weights within the composition.

### DETAILED DESCRIPTION OF THE INVENTION

As used herein, the term “indolocarbazole” is meant to refer to compounds having a core chemical structural formula:

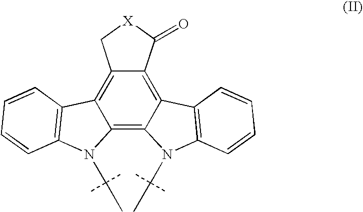

and substituted and fused cyclized derivatives thereof. These compounds are associated with the chemical property of being highly insoluble in aqueous systems.

As used herein, the term “emulsion” is intended to refer to a colloidal dispersion comprising water and organic components including hydrophobic (lipophilic) organic components. Generally, a traditional emulsion is comprised of oil droplets (>about 200 nm) dispersed in water, resulting in a milky white liquid which is not stable. The anatomy of a microemulsion (described below) is comprised of small bicontinuous channels of water and oil phase which also differs from the droplet shape of a classical emulsion.

The term “microemulsion,” as used herein, is intended to refer to a dispersion comprising water and organic components including hydrophobic (lipophilic) organic components, wherein the droplets or particles formed from the organic components have an average maximum dimension of less than about 200 nm. The term is also meant to describe and encompass compositions exhibiting certain characteristics or properties typically associated with microemulsions, including: spontaneous formation without high shear, lack of excessive heating needed for formation, thermodynamic stability, isotropic and optical clarity in molten form, no API crystals present in solid as measured by XRP diffraction (i.e. solid solution by XRPD), generally a range of about 15 to about 100 nm particle size as measured/observed by quasielastic light scattering data, and exhibition of bloom effect upon dilution into water—as are typically associated with microemulsions.

As used herein, the term “about” refers to a range of values from +10% of a specified value, and functional equivalents thereof unless otherwise specifically precluded. For example, the phrase “about 50 mg” includes ±10% of 50, or from 45 mg to 55 mg.

As used herein, “pharmaceutically acceptable”, within the context of describing vehicle or excipient ingredients, includes any ingredients that, within the scope of sound medical judgment, are suitable for oral administration and contact with the tissues of human beings and animals without excessive toxicity, irritation, allergic response, or other problem complications commensurate with a reasonable benefit/risk ratio.

In general, the invention provides a pharmaceutical composition comprising: an indolocarbazole compound present in a solubilized concentration of at least about 3% up to about 9% by weight of the total composition; a hydrophilic component; and water; wherein the composition is in the form of a microemulsion. In one embodiment, the indolocarbazole compound is [9S-(9α,10β,12α)]-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one, also referred to as lestaurtinib. In a preferred embodiment, the composition can further comprise a surfactant and antioxidant component.

Active ingredients that can be used in the invention include indolocarbazole compounds. Indolocarbazole compounds having the core unit structure as shown below in Formula (II)

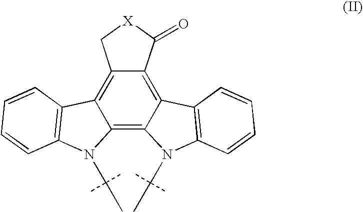

wherein X is O or N, and substituted and cyclized derivatives are generally known to be highly insoluble per se. Suitable indolocarbazole compounds that can be used include, but are not limited to, [9S-(9α,10β,12α)]-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one (CAS Registry Number 111358-88-4 and as shown in Formula (I) and also referred to under the generic name lestaurtinib, and the indolocarbazole compound known as K-252a.

It may be possible to use other therapeutic substances or compounds with poor aqueous solubility with the invention as well, provided such substances and compounds are chemically compatible in the composition of the invention and can substantially share the attributes and benefits associated with the invention.

The present invention provides a pharmaceutical composition which can also be characterized as a pharmaceutical fill composition suitable for encapsulated dosage forms. It is in the form of an encapsulated dosage form, such as a hard capsule, that the benefits and practical advantages of the invention can be fully realized. This is because the composition is prepared using an improved process that prepares a stable microemulsion having solubilized indolocarbazole compounds in significantly higher concentrations than previously accomplished for a relatively small total volume or weight. The pharmaceutical fill compositions prepared thusly are also suitable for encapsulation and which contain relatively high solubilized concentrations of indolocarbazole compounds in the form of a stable microemulsion.

The degree of optical transparency of a given volume of water containing a given amount of formulation gives a useful indication of particle size. This is because the particles scatter visible light, with the larger particles causing greater scattering. In general, the greater the optical transparency, the smaller the particle size. High optical transparency, i.e., bluish haze invisible or nearly invisible, generally indicates a particle size of less than 100 nm. A distinct bluish haze generally indicates a particle size from about 100 nm to about 400 nm. If particles fail to form, an increase in dilution ratio may be used to promote particle formation.

Whether a formulation according to the invention is a liquid, semi-solid, or solid at room temperature, can depend upon the selection of components and their properties, or other practical concerns such as commercial viability, administration comfort and frequency, and the like. For example, a semi-solid or solid formulation is convenient for manufacturing unit doses of indolocarbazoles in the form of a capsule, including both hard and soft capsules, e.g., mammalian- or fish-derived gelatin or hydroxypropylmethylcellulose (HPMC) capsule materials, and tablets. When the formulation contacts a fluid or liquid medium, e.g., gastrointestinal fluids, the formulation disperses into suspended particles in which the indolocarbazole is biologically available.

The enhanced solubilization result of indolocarbazole compounds achieved by the invention is attributable in part to the microemulsion formation process. It has been discovered that during the microemulsion formation stage of preparing compositions in accordance with the invention, the addition of water surprisingly facilitates and increases the solubilized concentration of indolocarbazoles, which are known to be highly insoluble in aqueous solutions.

### Process for Preparing

During the preparation of the composition according to the invention, the heated or molten composition state (i.e., the liquid stage) exhibits a level optical clarity which, as discussed herein above, is also associated with, or and indicator of, the extent of solubilization/lack of precipitation of the ingredients during the process. In certain embodiments of the invention, as the resultant composition returns to ambient temperature, e.g., storage environment conditions, the composition becomes increasingly opaque and quickly forming a semi-solid or solid composition (i.e., a “solid” stage). Nevertheless, despite the optical transition to opacity, the active indolocarbazole ingredient still remains in solution without significant crystallization or precipitation over the storage life of the composition.

Another aspect to the process of the invention is the discovery of the role of water within the process. Water is added during the admixture steps in the presence of the active indolocarbazole compound(s) which, in combination with the other ingredients, increasingly dissolves the indolocarbazole compound(s) in solution to achieve relatively high concentrations of the active ingredient in solubilized state.

Preferably, water is added during the period following the admixture step—wherein the active ingredient, e.g., indolocarbazole, has been combined with the hydrophilic ingredient(s) and other vehicle ingredients. It has been discovered that, within a defined range of active concentration between about 3% and about 9% total weight, there is a positive correlation between the addition of water and the amount of solubilized active ingredient achievable by the process of the invention. It is the addition of water during the microemulsion formation process that permits solubilized concentrations achievable greater than about 29 mg/g as with previous formulation efforts. The addition of water is associated with the formation of the microemulsion particle structures and can achieve active concentrations as much as about 3-fold as compared to compositions formed without using the invention.

One important aspect of the invention is that once the microemulsion composition has been created, excess additional water can then subsequently be removed without significantly adversely affecting the achieved concentration of solubilized active ingredient in the resultant microemulsion composition. In explaining the invention, it is therefore helpful to clarify that the role of water in the invention is discussed in two contexts—1) the amount of water added during the microemulsion formation process, and 2) the amount of water present in the resultant pharmaceutical composition.

During the process stage, the amount of water added can vary generally between about 1% and about 12%, and will vary according to the desired solubilized concentration of indolocarbazole. To achieve a solubilized concentration of lestaurtinib of 66 mg/g in the final composition, for example, the minimum amount of water added during the process would be about 6.5%. Again, there is a positive correlation between the amount of water added during the process and the achievable solubilized concentration of active according to the invention.

The amount of resultant water in the final microemulsion at the conclusion of the process can typically be less than the amount of water added during the process. This can be largely attributed to evaporation and expected water loss as may occur during the process, which occurs at elevated temperatures. Once the microemulsion composition has been formed, it is stable and the amount of water present in the resultant composition can be reduced to as low as 0.8% total composition weight. In the resultant composition, the amount of water present depends upon the desired indolocarbazole drug load, capsular material properties, and the like, and can vary. Hypothetically, it is envisioned by the inventors that one skilled in the art could desiccate or reduce the amount of water present in the resultant composition of the invention to achieve microemulsions with up to 10, 11 or 12 mg/g drug loads.

Generally, the amount of water in the prepared composition using lestaurtinib as the active indolocarbazole, for instance, can range between the minimum amount sufficient to produce solubilization for a given active amount up to an amount absent bloom effect and exhibiting optical clarity. Suitable amount of water for the invention can range from about 0.8% to about 50% total composition, preferably from about 3% to about 9%, and most preferably about 6% to about 8%. For manufacturing purposes, it is preferable to add a slight excess of water to accommodate expected water loss during processing. It will be understood by one skilled in the pharmaceutical manufacturing field that suitable water content for capsular dosage forms will need to account for the chemical interaction between water and the particular capsular material employed.

Overall, the water during the process can be added within the temperature range between the melting point of the excipients and below the boiling point of water. More typically, the water can be added at a temperature ranging from about 40° C. to about 100° C. Preferably, the water is added at a temperature of between about 50° C. to 65° C.

Minor departures below and above the above temperature range can be used. Further, it will be understood by one skilled in the art that variations of temperature used in the process of the invention can depend on melting points and/or stability of the excipients (e.g., PEG, surfactant, antioxidant), processing technique (e.g. extruder affords higher temperatures and use of high molecular weight (MW) PEGs), formation of impurities, degradation of active ingredient, and the like. The formation of a microemulsion, however, will be become increasingly difficult when the water addition step is performed beyond, i.e., above and below, the range of about 50° C. to about 65° C. temperature point. Given the description of the invention herein, one skilled in the pharmaceutical formulation art will know to adjust the temperature of the mixture accordingly to accommodate the particular melting point and chemical interactive properties of the individual and collective ingredients.

Alternatively, compositions wherein the vehicle components are all liquid at room temperature can be prepared by simply mixing the components without heating. The desired amount of active compound can be weighed out and dissolved in the mixture of inert components, without heating. Moderate heating, preferably less than 60° C., can be applied to hasten complete mixing of the inert components, and to hasten dissolution of the active compound.

In a further embodiment of the present invention, the active compound can be micronized in order to facilitate microemulsion formation. For example, milled or micronized active particles can be introduced into the process, thereby increasing surface area of the active, and increasing the speed of microemulsion formation in the process. Advantages include reduced processing time and reduced likelihood of active degradation.

Amounts of Indolocarbazole

One of the most important advantages associated with the invention is the ability to achieve concentration levels of active indolocarbazole, which in turn permits smaller total formulation or composition weights and/or volumes to be used to deliver the same therapeutically effective amount of active as compared to previous formulations. Furthermore, the invention achieves these relatively high concentrations while maintaining bioactivity of the active indolocarbazole. As described herein above, there is a correlation in the invention between the achievable solubilized concentration of active and the amount of water that can be used during the process of making the composition. The invention also achieves these concentrations without significant attenuation of the bioactivity of the active.

Active concentrations significantly greater than 30 mg/g (up to about 87 mg/g) are obtainable as compared to prior formulation efforts which achieved at most about 29 mg/g and were in the form of elevated volume SEDDS. These prior art formulations, thus, present substantially awkward dosage form sizes. For example, in a SEDDS formulation prepared using a vehicle composition having 25% PEG 400 and 25% PEG 1000 hydrophilic component and 50% MYRJ® 52 surfactant, the maximum solubility obtainable for lestaurtinib was about 29 mg/g. As a result of this upper solubility limit, encapsulated dosage forms could not be practically manufactured due to the fill volume capability limits for this formulation associated with the automated capsule manufacturing equipment. With these limitations, a maximum potency of 20 mg dosages is achievable in size 0 capsules (0.625 ml fill volume).

As a result of the process of the invention, however, a wide range of solubilized and greater concentrations of indolocarbazoles are achievable. Concentrations as high as 87 mg/g, or about 9% total composition weight, have been achieved with the active lestaurtinib using the invention. Put another way, a significantly greater concentration of indolocarbazole compound can be achieved in solubilized form for a given fill volume. Among the practical advantages and benefits associated with the instant invention include the ability to achieve the same required or desired daily dosage and bioavailability of pharmaceutically active indolocarbazole compounds, such as lestaurtinib, with smaller capsule sizes and/or fewer capsules needed per unit time, and administration episodes, thereby increasing convenience, patient comfort and potentially patient compliance. In general, the amount of indolocarbazole compound can be present in an amount from about 3% to about 9% total composition weight. Even more preferably, the amount of indolocarbazole compound can be present in an amount from about 5% to about 7% total composition weight; most preferably, the amount can be about 6.6% composition weight.

### Hydrophilic Polymer Component

The present composition comprises a hydrophilic polymer component. Suitable hydrophilic ingredients that can be used as the hydrophilic polymer component include a variety of pharmaceutically acceptable hydrophilic agents that participate in the formation of the microemulsion, permit the accomplishment of the high levels of solubilized active ingredient, and are chemically compatible with the capsular material of the dosage form.

In general, suitable hydrophilic polymers (i.e., two or more repeating monomer units) include, but are not limited to, pharmaceutically acceptable and water soluble polymers such as polyethylene glycols, methoxypolyethylene glycols, polyvinyl alcohols, polyvinyl pyrrolidones, and the like. The hydrophilic polymer component can also include combinations or mixtures of pharmaceutically acceptable and water soluble polymers as well.

As used herein, “polyethylene glycol” or “PEG” means a liquid or solid polymer of the general formula H(OCH₂CH₂)ₙOH, wherein n is at least 4. In certain embodiments, the hydrophilic component is a polyethylene glycol or a mixture of polyethylene glycols. Polyethylene glycols that can be used can include a wide range of molecular weights. In general, suitable polyethylene glycols that can be used with the invention include those from about PEG 400 to about PEG 8000, preferably PEG 400 to about PEG 1500, most preferably PEG 1000. Polyethylene glycols that can be used include, but are not limited to, PEG-400, PEG-600, PEG-1000, PEG-1450, PEG-1500, PEG-3350, or PEG-4600.

The composition of the invention can include one PEG or, alternatively, a mixture of two or more of the aforementioned polyethylene glycols. Representative mixtures include PEG-400/PEG-1000, PEG-400/PEG-1450, PEG-600/PEG-1000, PEG-600/PEG-1450.

The amount of hydrophilic polymer component, e.g., polyethylene glycol to be used in the composition can vary provided a microemulsion is formed. In general, the amount of hydrophilic component can be present in an amount from about 10% to about 95% per total composition, preferably from about 30% to about 50%. Even more preferably, the amount of hydrophilic component can be present in an amount from about 35% to about 45% per total composition, most preferably about 42%.

### Surfactants:

It is possible to achieve the advantages of the invention, e.g., high solubilized concentrations of indolocarbazole compounds, without the use of a surfactant. The compositions of the present invention, however, preferably include at least one surfactant. The use of a surfactant can provide benefits in regard to dissolution or delivery stability. Suitable surfactants include, but are not limited to, nonionic, anionic and cationic surfactants, and combinations thereof.

Examples of suitable anionic surfactants that can be used, include, but are not limited to, sodium laurylsulfate or sodium dodecylsulfate. Examples of suitable cationic surfactants that can be used include, but are not limited to, cetyl trimethyl ammonium bromide (C-TAB). Examples of nonionic surfactants that can be used include, but are not limited to, polyoxyethylene stearates, such as polyoxyl 40 stearate (e.g., MYRJ® 52).

In addition to the above suitable surfactants for use in the invention include, but are not limited to, polyoxyethylene stearates, polyoxyethylene castor oil, polyoxyethylene sorbitan fatty acid esters (sorbitans), saturated polyglycolized glycerides, fatty acid esters of polyethylene glycol, hydroxylated lecithins, medium chain monoglycerides, medium chain fatty acid esters, polyethylene/propylene glycol copolymers, polyethylene glycol stearate, d-α-tocopheryl polyethylene glycol succinate, poloxyl stearate (e.g., Myrj® 52) and poloxyl castor oil. Polyoxyethylene sorbitan fatty acid esters (polysorbates) are non-ionic surfactants (detergents) that may consist of a mixture of fatty acids. Commercially available examples are Tween® 20 (polyoxyethylene (20) sorbitan monolaurate), Tween® 40 (polyoxyethylene (20) sorbitan monopalmitate), and Tween® 80 (polyoxyethylene (20) sorbitan monooleate). Non-ionic surfactants are preferred.

Examples of other useful surfactants are saturated polyglycolized glycerides consisting of mono-, di-, or triglycerides; di-fatty acid esters of polyethylene glycol, e.g., Gelucire® 44/14; hydroxylated lecithins, e.g., Centrolene® A; medium chain monoglycerides, e.g., glyceryl monocaprylate (Imwitor® 308, Capmul® MCM C-8); medium chain monoglycerides and diglycerides, e.g., glyceryl caprylate/caprate (Capmul® MCM); polyethylene/propylene glycol copolymers; block copolymers of ethylene oxide and propylene oxide (e.g., Poloxamer 188, Pluronic® F-68); ethoxylated castor oil (e.g., Cremophor® EL); and ethoxylated hydroxystearic acid (e.g., Solutol® HS 15). Some surfactants are solid or semisolid at room temperature, e.g., Poloxamer 188, glyceryl monocaprylate, and Gelucire® 44/14. Additional surfactants are those found in The Handbook of Pharmaceutical Excipients, 2nd Ed., published by The Pharmaceutical Press, London and American Pharmaceutical Association (1994), a common text in the field, which is hereby incorporated by reference in its entirety.

In certain embodiments, the surfactant can be a polyoxyl stearate. In a further embodiment, the polyoxyl stearate can be polyoxyl 40 stearate (MYRJ® 52).

The amount of surfactant used in the invention, when present, can vary provided the amount is sufficient to participate in the formation and/or stabilization of the microemulsion. In general, the amount of surfactant if used is present in an amount from about 0.1% to about 50%—depending upon the particular surfactant employed. When MYRJ® 52 is used, the amount can be between about 5% and about 50% by weight of the total composition, preferably between 10% and 45% by weight of the total composition. Even more preferably, the amount can be between about 35% and 45% by weight of the total composition. Most preferably, the amount can be about 42% by weight of the total composition.

### Antioxidants

In additional embodiments of the invention, a suitable antioxidant is included as a composition ingredient. As used herein, “antioxidant” is intended to indicate any substance useful to retard deterioration by oxidation or to inhibit reactions promoted by oxygen or peroxides. The use of an antioxidant is important to the stability of encapsulated dosage forms by reducing both oxidation of formulation ingredients as well as capsule material or shell degradation caused by the presence of oxidation impurities. The presence of an antioxidant within the composition of the invention, however, is contingent upon the need for one, i.e., the susceptibility of the active ingredient and/or excipient to chemical oxidation and consequential generation of impurities therefrom.

The main oxidation mechanisms for organic molecules include reaction with peroxides, catalysis by transition metals, autoxidation and light-initiated oxidation.

Selection of Antioxidants

Various antioxidants were evaluated for their effectiveness in relation to the composition of the invention. In an initial experiment, capsules containing a lestaurtinib composition prepared according to the invention were placed on accelerated stability conditions. Several impurities were determined (LC-MS) to be related to the oxidation of lestaurtinib. Subsequently, a forced degradation experiment was performed using the radical initiator azoisobutyronitrile (AIBN) adapted for use with lestaurtinib. This was based on the fact that AIBN radical initiates the autoxidation mechanism that the majority of active ingredients follow for oxidation. The forced degradation experiment showed excellent impurities correlation between the active ingredient treated for 48 hours at 40° C. in the presence of AIBN and thermally generated impurities (3-6 month at 30° C.).

Using AIBN as a screening tool, a series of microemulsions prepared in accordance with the invention were formulated using different antioxidants to identify suitable solutions for preventing degradation of the composition. The first experiment used thirteen formulations using several classes of antioxidants (i.e., oxygen scavengers, sacrificial antioxidants and H-atom donors) to determine which reaction pathway was critical for disrupting the oxidation process. From this first experiment, several antioxidants were identified as efficacious: ascorbic acid, vitamin E, BHA and BHT.

A second experiment was then performed based on the information obtained from the first experiment. The second experiment evaluated combinations of antioxidants from different classes, different isomers of the same antioxidants, and mixtures of water-soluble and oil-soluble antioxidants. The results showed that the combination of ascorbic acid (water soluble) and either vitamin E (preferred) or BHA showed improvement over either the BHA or vitamin E alone. It was further observed that the addition of a peroxide scavenger such as potassium metabisulfite (KMBS) decreased the overall impurity levels in the final formulation as well.

Based on the results of the above experiments, the preferred antioxidants and combinations were identified for the invention. Preferably, the antioxidant used with the invention comprises a combination of an H-atom donor (which interfere with the propagation step by quenching the radical) or a sacrificial oxidant (a compound that is more readily oxidized than the active ingredient and inteferes with the initiation step). Examples of H-atom donor antioxidants include, but are not limited to, butylhydroxyanisole (BHA), butylhydroxytoluene (BHT) and propyl gallate. Examples of sacrificial oxidants include, but are not limited to, vitamin E, ascorbic acid, ascorbyl palmitate and sodium ascorbate, and salts and esters thereof.

Preferably and to achieve optimal stability of the encapsulated dosage form made according to the invention, a combination of oil-soluble and water soluble antioxidants is used. This is the case with the combination of antioxidant vitamin E and ascorbic acid.

Other antioxidants can be used as well, such as oxygen scavenger antioxidants. Suitable oxygen scavenger antioxidants include sulfite salts and metabisulfite salts.

Antioxidants which can be used for the invention include vitamin E, ascorbic acid, KMBS, ascorbyl palmitate, sodium ascorbate, BHA, BHT, and combinations thereof. Preferred combinations of antioxidant ingredients for use with the invention include: 1) vitamin E, ascorbic acid, and KMBS; 2) vitamin E, ascorbyl palmitate, ascorbic acid, and KMBS; 3) vitamin E, ascorbyl palmitate, sodium ascorbate and KMBS; 4) vitamin E, ascorbyl palmitate and ascorbic acid; 5) vitamin E and ascorbyl palmitate; and 6) BHA, ascorbic acid, and KBMS. More preferred are 1) vitamin E (0.08%), ascorbic acid (0.1%) and KMBS (potassium metabisulfite salt) (0.05-0.1%); 2) vitamin E (0.08%), ascorbyl palmitate (0.1%), ascorbic acid (0.1%) and KMBS (0.05-0.2%); and 3) BHA (0.02%), ascorbic acid (0.1%) and KBMS (0.05-0.1%). Even more preferred are 1) vitamin E (0.0750%), ascorbyl palmitate (0.1000%), ascorbic acid (0.1000%) and KMBS (0.2000%); 2) vitamin E (0.0750%), ascorbyl palmitate (0.1000%), sodium ascorbate (0.1250%) and KMBS (0.2000%); 3) vitamin E (0.1500%), ascorbyl palmitate (0.2000%) and ascorbic acid (0.0500%); and 4) vitamin E (0.1500%) and ascorbyl palmitate (0.2000%). Even more preferred antioxidant ingredients for use with the invention include vitamin E in combination with ascorbic acid. Most preferred is vitamin E, ascorbyl palmitate, ascorbic acid and KMBS.

The amount of antioxidant, if present, can vary. In general, an amount of total antioxidant can be present from 0% to about 2%. In one embodiment wherein the invention is in the form of an encapsulated hard gelatin pharmaceutical fill composition as a 20 mg capsule containing 66 mg/g lestaurtinib, the amount of antioxidant component can be present in an amount of about 0.5% by weight of the total composition.

### Additional Excipients

In further embodiments, the compositions of the present invention can optionally include other pharmaceutically acceptable secondary ingredients in the vehicle or excipient component provided they do not interfere or significantly attenuate the benefits associated with the invention. The use of such media and agents for pharmaceutical active substances is well known in the art, such as in Remington: The Science and Practice of Pharmacy, 20th ed.; Gennaro, A. R., Ed.; Lippincott Williams & Wilkins: Philadelphia, Pa., 2000. Examples of such pharmaceutically acceptable secondary ingredients include, but are limited to, coloring agents, flavoring agents, solvents, dispersion media, coatings, antibacterial and antifungal agents, isotonic and absorption delaying agents, and the like. Except insofar as any conventional media or agent is incompatible with the active ingredient, its use in the therapeutic compositions is contemplated. Supplementary active ingredients can also be incorporated into the compositions. Suitable pharmaceutically acceptable excipients for use in the present invention include, but are not limited to, magnesium aluminometasilicate, microcrystalline cellulose, lactose, sodium starch glycolate and magnesium stearate.

### Capsular Dosage Forms

The invention includes a dosage form, in particular an oral capsular dosage form, comprising the pharmaceutical composition as a fill formulation for encasement and encapsulation in a capsular material. While a variety of oral dosage forms can be used to deliver indolocarbazole compounds such as lestaurtinib, suitable dosage forms that can be used must accommodate the physical and chemical properties of the pharmaceutical composition prepared in accordance with the invention. It should also be noted that additional active ingredients can be included provided such do not substantially interfere with or or attenuate the advantageous properties associated with the inventive composition. Because the advantages of the invention are fully realized within the context of formulations for oral/gastro-intestinal route administration, the invention is particularly useful in formulations for dosage forms of the hard capsule variety.

A variety of capsular materials can be used to prepare the dosage form of the invention, provided they are suitable for encapsulation of molten liquid fill compositions and the capsule material is chemically compatable with the fill composition. Preferred for use with the composition of the invention is gelatin-preferably hard gelatin capsule materials. Gelatin capsule materials can be composed of mammalian-derived or fish-derived gelatin. Other capsule materials can be used as well, such as hydroxypropylmethylcellulose (HPMC) capsule materials. In a less preferred embodiment, soft capsule dosage forms can be prepared, provided the fill formulation has been modified to be chemically compatible with the capsule material and the formulation maintains the advantages associated with the invention. Other capsule materials that can be used include various cellulose and cellulose-derived materials, iota-carrageenan-containing capsule materials, and the like.

Capsular dosage forms, such as hard capsules, containing the pharmaceutical fill composition of the invention can be prepared using a variety of well-known techniques and equipment readily available to one skilled in the pharmaceutical encapsulation field. In addition to readily available hard and soft capsule manufacturing resources, examples of hard (gelatin) capsule manufacturing equipment and processes are described in, for example, U.S. Pat. Nos. 4,281,763; 4,325,761; 4,408,641; 4,917,885; 5,419,916; 6,752,953—the texts of which are incorporated herein by reference.

In general, suitable hard capsule and in accordance with one technique, the hard capsule shell is prepared in advance and is composed of two separated interfitting portions. A capsule filling apparatus positions the two interfitting halves relative to one another while filling the receiving shell portion with the liquid, molten formulation or powder, and subsequently fits the remaining shell to enclose the contents of the capsule to form the resultant filled capsule. Various hard capsule sealing techniques can be used as well, which are especially useful with non-powdered fill compositions.

As discussed herein above, as a result of the inventive achievement of significantly higher solubilized concentrations of the active ingredient, a relatively smaller fill volume can be used to deliver the same dosage amount of the active ingredient, and thereby smaller capsule sizes can be used to accomplish the same bioavailability of the active. In a multiple dosage unit scenario, fewer and/or smaller capsules can be administered to the recipient.

### Methods of Treatment/Therapeutic Utility

The invention includes a method of inhibiting receptor-tyrosine kinase in a recipient comprising administering to such recipient receptor-tyrosine kinase-inhibiting amount of an indolocarbazle compound as formulated in accordance with the instant invention. As used herein, the term “recipient” is meant to include mammals, e.g., humans, to which the composition or dosage form prepared according to the invention is administered.

The present invention further provides for a method of treating a disease and/or condition in a subject in need such treatment comprising administering to said subject a therapeutically effective amount of an indolocarbazole compound within the composition of the present invention. In accordance with the various treatments and therapeutic effects known to be associated with indolocarbazoles and the compound of Formula (I), these compounds may be useful for treating a variety of therapeutic indications to those described in the patents and applications identified herein above in the background section.

According to the invention, a given method of treatment comprises administration of a “therapeutically effective amount”—the term which as used herein is meant to refer to the amount determined to be required to produce the physiological effect intended and associated with a given drug, as measured according to established pharmacokinetic methods and techniques, for the given administration route. In the context of the dosage form of the invention, the term refers to a context of oral administration via the gastrointestinal route. Appropriate and specific therapeutically effective amounts can be readily determined by the attending diagnostician, as one skilled in the art, by the use of conventional techniques. The effective dose will vary depending upon a number of factors, including the type and extent of progression of the disease or disorder, the overall health status of the particular patient, the relative biological efficacy of the compound selected, the formulation of the active agent with appropriate excipients, and the route of administration. Typically, the compounds are administered at lower dosage levels, with a gradual increase until the desired effect is achieved.

For example, the compounds of the present invention may be useful for the treatment of a wide variety of cancers, including, for example, carcinomas of the pancreas, prostate, breast, thyroid, colon, and lung; malignant melanomas; glioblastomas; neuroectodermal-derived tumors including Wilm's tumor, neuroblastomas, and medulloblastomas; and leukemias including, but not limited to, acute myeloid leukemia (“AML”), chronic myeloid leukemia (“CML”), acute lymphocytic leukemia (“ALL”), and chronic lymphocytic leukemia (“CLL”); pathological conditions of the prostate, such as prostatic hypertrophy or prostate cancer; carcinomas of the pancreas, such as pancreatic ductal adenocarcinoma (PDAC); hyperproliferative disorders, such as proliferative skin disorders including actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma and various forms of psoriasis, including psoriasis vulgaris and psoriasis eosinophilia. Preferably, the invention includes a method of treating acute myeloid leukemia (AML), and myeloproliferative disorders (MPDs) including chronic mylogenous leukemia (CML), polycythemia vera (PV), essential thrombocythemia (ET), chronic idiopathic myelofibrosis (CIMF/AMM), chronic eosinophilic leukemia (CEL), chronic neutrophilic leukemia (CNL), and hypereasinophilic syndrome (HEL). More preferably, the invention includes a method of treating acute myeloid leukemia (AML).

Typical dose ranges can be from about 0.01 mg/kg to about 100 mg/kg of body weight per day, or a dose from about 0.01 mg/kg to 10 mg/kg of body weight per day. Daily doses for adult humans includes about 20, 25, 30, 35, 40, 45, 50, 55, 60, 65, 70, 75, 80, 90, 100, 120, 140, 160 and 200 mg, and an equivalent dose in a human child. The compounds may be administered in one or more unit dose forms, and may be administered one to four times daily, including twice daily (“bid”). The unit dose ranges from about 1 to about 400 mg administered one to four times a day, or from about 10 mg to about 200 mg bid, or 20-80 mg bid, or 60-100 mg bid or from about 40, 60, 80, or 100 mg bid. Alternatively, the dosage may also be in the form of a liquid, in a concentration of between about 30 mg/g and about 90 mg/g. The liquid dosage forms may then include the equivalent of the doses (mg) described above. Conventional pharmacokinetic calculations readily available to those skilled in the art can be used to formulate dose to achieve the desired serum concentration of active.

The following examples further illustrate the invention and are not intended to be construed as necessarily limiting the invention.

### EXAMPLES

The present invention provides a process for preparing a microemulsion composition containing one or more indolocarbazole compounds and a resulting pharmaceutical composition prepared by the process. Specifically, a process for preparing a microemulsion composition comprising the indolocarbazole compound of Formula (I), lestaurtinib, is described.

In general, the microemulsion composition of the present invention can be formed by combining and melting the appropriate amounts of the hydrophilic agent, and in particular, the PEG(s) and the surfactant(s), to form an initial excipient solution. The active agent can then be added to the excipient solution and stirred with heating to form a slurry. After 20 minutes of stirring, water can be added to the slurry to yield the microemulsion composition. This process can be accomplished in a shorter time frame than typical microemulsion formation processes, which decreases the amount of degradation of the formulation due to processing. Another advantage of the instant process is that excessive shear is not required.

The encapsulation of the formulation to obtain the final dosage form is performed by filling the desired amount of molten formulation (e.g., liquid stage) into the desired capsule conveyance (hard or soft gelatin) under the appropriate conditions for manufacture. For instance, one manufacturing procedure for a hard gelatin 20 mg capsule involves filling 303 mg of a 66 mg/g formulation into the cavity of a hard gelatin capsule. The cap of the capsule assembly is then fitted onto or coupled to the other capsule shell portion, and the encapsulated dosage form is allowed to cool to ambient temperature on collecting trays. Once the contents have solidified (e.g., “solid” stage), the coupled capsule shell can be sealed (banded) by applying a band of gelatin around the junction of the two shell halves. This process can be performed either manually (using hand-operated equipment) or as a fully automated process (using automated production equipment) which can produce as many as 200,000 capsules or more.

### Example 1

### Preparation of 100 g Batch Microemulsion Composition Containing 66 mg/g Lestaurtinib

A microemulsion composition according to the invention was prepared according to the following procedure. A 100.0 g stock solution of a 1:1 mixture by weight of PEG-1000 and MYRJ® 52 was prepared by weighing 50.0 g of MYRJ® 52 into a clean 250 mL beaker. A magnetic stir bar was added, and 50.0 g of molten PEG-1000 was weighed into the beaker. The mixture was then stirred on a hot plate at approximately 55° C. until a uniform solution was obtained.

The excipient solution was then used to prepare a 100.0 g batch of a microemulsion composition containing the compound of Formula (I). The microemulsion was prepared by first weighing 6.6 g of Formula (I) compound into a clean glass beaker that had been outfitted with a magnetic stir bar. The beaker was then charged with 85.4 g of the liquid excipient solution and allowed to stir at approximately 55° C. After 5 minutes, 8.0 g of sterile water was added to the slurry and stirred on a hot plate heated to approximately 55° C. until a homogeneous solution was obtained (at approximately 10 minutes). The resulting 100 g batch of microemulsion composition had a concentration of 66 mg/g indolocarbazole (of Formula (I)). The final capsule formulation was prepared by filling 303 mg (275 μl) of this solution into a size 1 gelatin capsule. The resulting 20 mg capsule had the following formula:

TABLE 1
Capsule Fill Formulation 66 mg lestaurtinib

| Ingredient | Amount (mg) | | % amount |
|---|---|---|---|
| Lestaurtinib | 20.0 | | 6.6 |
| PEG-1000 | 129.4 | | 42.7 |
| MYRJ ® 52 | 129.4 | | 42.7 |
| Water | 24.2 | | 8.0 |
| Total: | 303.0 | mg | 100.0% |

It is possible to modify the formula of Example 1, or any composition of the invention, to include antioxidant ingredients to increase the oxidative stability of the active pharmaceutical ingredient; for example vitamin E, ascorbyl palmitate, ascorbic acid, sodium ascorbate and KMBS, as shown in Tables 1a, 1b, 1c and 1d below.

### Example 1a

### Preparation of 100 g Batch Microemulsion Composition Containing 66 mg/g Lestaurtinib with the Addition of Antioxidants

A microemulsion composition according to the invention was prepared according to the following procedure. A 100.0 g stock solution of a 1:1 mixture by weight of PEG-1000 and MYRJ® 52 was prepared by weighing 50.0 g of MYRJ® 52 into a clean 250 mL beaker. A magnetic stir bar was added, and 50.0 g of molten PEG-1000 was weighed into the beaker. The mixture was then stirred on a hot plate at approximately 55° C. until a uniform solution was obtained.

The excipient solution was then used to prepare a 100.0 g batch of a microemulsion composition containing the compound of Formula (Table Ib). The microemulsion was prepared by first weighing 6.6 g of Formula (I) compound into a clean glass beaker that had been outfitted with a magnetic stir bar. The beaker was then charged with 84.9 g of the liquid excipient solution, 0.075 g (7.5 mg) of vitamin E, 0.1 g of ascorbyl palimitate and 0.125 g of sodium ascorbate and allowed to stir at approximately 55° C. After 5 minutes, a solution comprised of 8.0 g of sterile water and 0.2 g of potassium metabisulfite was added to the slurry and stirred on a hot plate heated to approximately 55° C. until a homogeneous solution was obtained (at approximately 10 minutes). The resulting 100 g batch of microemulsion composition had a concentration of 66 mg/g indolocarbazole (of Formula (Table Ib)). The final capsule formulation was prepared by filling 303 mg (275 μl) of this solution into a size 1 gelatin capsule. The resulting 20 mg capsule had the following formula:

TABLE 1a
Capsule Fill Formulation 66 mg lestaurtinib including vitamin E,
ascorbyl palmitate, ascorbic acid and KMBS

| Ingredient | Amount (mg) | | % amount |
|---|---|---|---|
| Lestaurtinib | 20.00 | | 6.6 |
| PEG-1000 | 128.66 | | 42.4625 |
| MYRJ ® 52 | 128.66 | | 42.4625 |
| Vitamin E | 0.23 | | 0.0750 |
| Ascorbyl Palmitate | 0.30 | | 0.1000 |
| Ascorbic Acid | 0.30 | | 0.1000 |
| KMBS | 0.61 | | 0.2000 |
| Water | 24.24 | | 8.0000 |
| Total: | 303.0 | mg | 100.0% |

TABLE 1b
Capsule Fill Formulation 66 mg lestaurtinib including vitamin E,
ascorbyl palmitate, sodium ascorbate and KMBS

| Ingredient | Amount (mg) | | % amount |
|---|---|---|---|
| Lestaurtinib | 20.00 | | 6.600 |
| PEG-1000 | 128.62 | | 42.450 |
| MYRJ ® 52 | 128.62 | | 42.450 |
| Vitamin E | 0.23 | | 0.075 |
| Ascorbyl Palmitate | 0.30 | | 0.100 |
| Sodium Ascorbate | 0.38 | | 0.125 |
| KMBS | 0.61 | | 0.200 |
| Water | 24.24 | | 8.000 |
| Total: | 303.0 | mg | 100.0% |

TABLE 1c
Capsule Fill Formulation 66 mg lestaurtinib including vitamin E,
ascorbyl palmitate and ascorbic acid

| Ingredient | Amount (mg) | | % amount |
|---|---|---|---|
| Lestaurtinib | 20.00 | | 6.60 |
| PEG-1000 | 128.775 | | 42.50 |
| MYRJ ® 52 | 128.775 | | 42.50 |
| Vitamin E | 0.45 | | 0.15 |
| Ascorbyl Palmitate | 0.61 | | 0.20 |
| Ascorbic Acid | 0.15 | | 0.05 |
| Water | 24.24 | | 8.00 |
| Total: | 303.0 | mg | 100.0% |

TABLE 1d
Capsule Fill Formulation 66 mg lestaurtinib including vitamin E,
and ascorbyl palmitate

| Ingredient | Amount (mg) | | % amount |
|---|---|---|---|
| Lestaurtinib | 20.00 | | 6.600 |
| PEG-1000 | 128.85 | | 42.525 |
| MYRJ ® 52 | 128.85 | | 42.525 |
| Vitamin E | 0.45 | | 0.150 |
| Ascorbyl Palmitate | 0.61 | | 0.200 |
| Water | 24.24 | | 8.000 |
| Total: | 303.0 | mg | 100.0% |

The following examples may be prepared by using a similar methodology and by calculating the desired amounts of the desired components.

### Example 2a

### Preparation of a 5.6 g Batch of a 71 mg/mL Lestaurtinib Microemulsion (40 mg Dose Capsule)

Initially, 4.6 g of a 50/25/25 (wt %) molten blend of MYRJ®52/PEG-400/PEG-1000 was added to a scintillation vial outfitted with a magnetic stir bar on a hot plate set to a temperature of about 65° C. Once the solution was uniform, 0.40 g (400 mg) of lestaurtinib was added. The resulting slurry was then mixed for a period of 15 minutes to ensure that any aggregated active ingredient was broken up. Using a micropipette, 600 mg of DI water (density adjusted) was added and the formulation was stirred until a homogenous mixture resulted (approximately 1 minute). The formulation prepared is set forth in the following table.

TABLE 2
40 mg Lestaurtinib Formulation

| Ingredient | Amount mg | Amount % |
|---|---|---|
| Lestaurtinib | 400 | 7.14 |
| PEG-400 | 1150 | 20.54 |
| PEG-1000 | 1150 | 20.54 |
| MYRJ 52 | 2300 | 41.07 |
| Water | 600 | 10.71 |
| Total: | 5400 | 100.0% |

### Example 2b

Using a process similar to that set forth above in Example 2a, the following formulation was prepared.

TABLE 3
48.3 mg/ml*

| Ingredient | Amount mg | Amount % |
|---|---|---|
| Lestaurtinib | 400 | 4.00 |
| PEG-400 | 2325 | 23.25 |
| PEG-1000 | 2325 | 23.25 |
| MYRJ ® 52 | 4650 | 46.50 |
| Water | 302 | 3.00 |
| Total: | 10002 | 100.0% |

*This formulation was formulated to be 40 mg/g but by HPLC assay it was found to be 48.3 mg/ml concentration due to the 1.1 g/ml density and loss of some water often observed with the invention.

### Example 2c

Using a process similar to that set forth above in Example 2a, the following formulation was prepared.

TABLE 4
70.8 mg/ml*

| Ingredient | Amount mg | Amount % |
|---|---|---|
| Lestaurtinib | 600 | 6.00 |
| PEG-400 | 2189 | 21.89 |
| PEG-1000 | 2189 | 21.89 |
| MYRJ ® 52 | 4379 | 43.79 |
| Water | 643 | 6.43 |
| Total: | 9400 | 100.0% |

*This formulation was formulated to be 60 mg/g but by HPLC assay it was found to be 70.8 mg/ml concentration due to the 1.1 g/ml density and loss of some water often observed with the invention.

### Example 2d

Using a process similar to that set forth above in Example 2a, the following formulation was prepared.

TABLE 5
81.6 mg/ml*

| Ingredient | Amount mg | Amount % |
|---|---|---|
| Lestaurtinib | 700 | 7.00 |
| PEG-400 | 2122 | 21.22 |
| PEG-1000 | 2122 | 21.22 |
| MYRJ ® 52 | 4243 | 42.43 |
| Water | 814 | 8.14 |
| Total: | 10001 | 100.0% |

*This formulation was formulated to be 71 mg/g but by HPLC assay it was found to be 81.6 mg/ml concentration due to the 1.1 g/ml density and loss of some water often observed with the invention.

### Example 3

### Water Versus Drug Load Concentrations

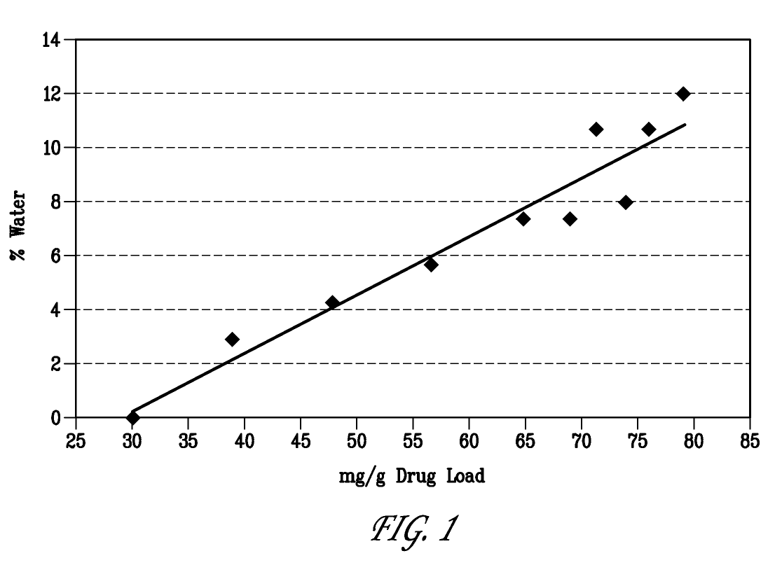

In order to demonstrate the relationship between the addition of water during the process of preparing the microemulsion composition of the invention and corresponding increases in solubilized indolocarbazole compound, the following experiment was performed. A series of ten mixtures were prepared using the methods of the invention containing similar excipient components and hydrophilic ingredients (50/50 wt/wt blend of PEG-1000 and PEG-400 and surfactant (MYRJ®52) but which varied the amount of solubilized indolocarbazole compound (lestaurtinib) from about 3% by weight to about 8.00% by weight. Subsequently, water was added until a clear, non-precipitating microemulsion was formed. The water content was determined along with mg of drug (lestaurtinib) per total weight of the composition. The resulting data was recorded and plotted by graph (see FIG. 1).

As can be seen from the data depicted in the graph of FIG. 1, it was observed that between added water content between about 3% and about 12%, a microemulsion can be formed with the active ingredient at the given levels of the hydrophilic component and the surfactant. The data also indicated that drug loads ranging from about 30 mg/g to about 80 mg/g can be prepared.

### Example 4

### Comparative Illustration of Microemulsion Formation and Opacity

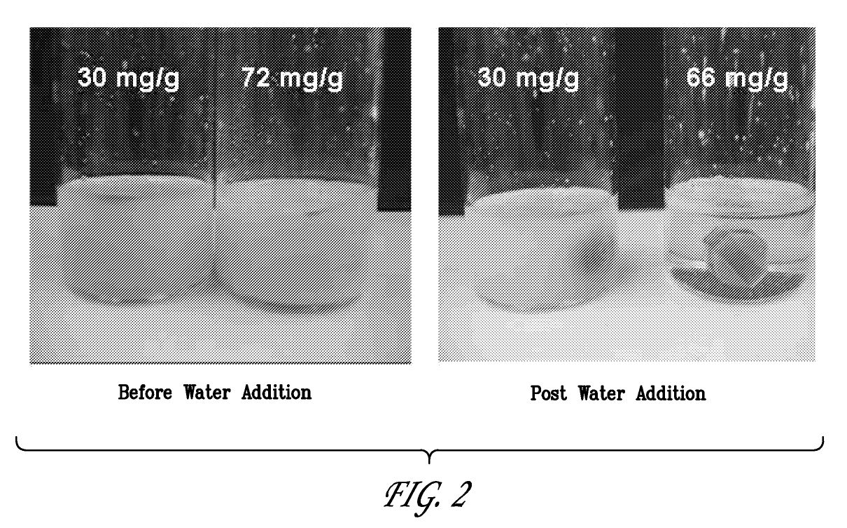

To illustrate the resulting solubility and clarity phenomenon associated with the invention, two compositions were prepared—each composition being a microemulsion containing the vehicle formula 25%/25%/50% (wt %) PEG-400/PEG-1000/MYRJ®52—with one composition containing 30 mg/g concentration of lestaurtinib and the second composition containing 72 mg/g pre-water addition. Both of the compositions were observed at two general stages—first each of the two compositions were observed without the presence of water in the microemulsion formation process. At the second stage, the 30 mg/g composition without water added appears alongside the 72 mg/g composition following the addition of 8% water using the microemulsion formation process of the invention (thus resulting in a final lestaurtinib concentration of 66.7 mg/g). As discussed herein, observed clarity and lack of precipitation of the active indolocarbazole compound are indicators of successful solubilized concentrations in the molten state of the composition. The two observed stages were photographed and the photographs appear in FIG. 2.

As can be seen from the photographs in FIG. 2, the 30 mg/g composition still appears opaque in the absence of added water. However, the function and effect of water in the composition containing a substantially greater concentration of active compound (starting at 72 mg/g and corresponding post-water 66 mg/g concentration) is readily visible. The clarity of the 66 mg/g composition indicates the successful achievement of solubilized active in microemulsion form when prepared in accordance with the invention.

### Example 5

### Preparation of Lestaurtinib Formulation Using Anionic Surfactant

A clean glass scintillation vial was charged with 0.66 g of lestaurtinib. PEG-1000 in an amount of 8.45 g was then added to the vial in molten liquid state. A magnetic stir bar was added alongside 0.09 g of sodium dodecyl sulfate, and the resultant mixture was stirred on a hotplate set at temperature of approximately 60° C. After mixing for a period of about 5 minutes, 0.80 g of DI water was added. The mixture was then stirred in a capped scintillation vial on a hotplate set at approximately 60° C. until a clear homogenous solution was obtained (a period of about 10 minutes).

### Example 6

### Preparation of Lestaurtinib Formulation Using Cationic Surfactant

A clean glass scintillation vial was charged with 0.66 g of lestaurtinib. PEG-1000 in an amount of 8.45 g was then added to the vial in molten liquid state. A magnetic stir bar was added alongside 0.09 g of cetyl trimethyl ammonium bromide, and the resultant mixture was stirred on a hotplate set at temperature of approximately 60° C. After mixing for a period of about 5 minutes, 0.80 g of DI water was added. The mixture was then stirred in a capped scintillation vial on a hotplate set at approximately 60° C. until a clear homogenous solution was obtained (a period of about 10 minutes).

### Example 7

### Dilution Stability Letaurtinib Formulations containing Anionic and Cationic Surfactants

To evaluate their dilution behavior, both of the formulations prepared according to the above examples were diluted 1 to 10 into room temperature DI water. Upon dilution, both formulations changed from clear homogenous solutions to opaque milky-white emulsions with no apparent signs of precipitation of the active ingredient lestaurtinib. No post-dilution changes were observed in the diluted samples following unagitated storage for a period of 18 hours at ambient temperature conditions.

### Example 8

### Effect of Surfactant Amounts on Particle Size of Microemulsion Formulation

The relationship between varying percent (% total composition) surfactant amounts and particle size was evaluated. More specifically, varying amounts of polyoxyethylene stearate (MYRJ® 52) were formulated in the vehicle composition with PEG-400 and PEG-1000 as follows:

TABLE 6
Variable Surfactant % Amount Vehicle Formulation

| | | % PEG-400/PEG-1000 | % | |
|---|---|---|---|---|
| Sample | % MYRJ ® | (50/50 w/w) | Lestaurtinib | % Water |
| 5% | 4.4 | 83.7 | 6.6 | 5.2 |
| 10% | 8.7 | 77.9 | 6.5 | 7.0 |
| 15% | 13.0 | 73.9 | 6.5 | 6.5 |
| 20% | 17.3 | 69.2 | 6.5 | 7.0 |
| 25% | 21.8 | 65.5 | 6.5 | 6.1 |
| 30% | 26.2 | 61.1 | 6.6 | 6.1 |
| 35% | 30.4 | 56.5 | 6.6 | 6.5 |
| 40% | 34.6 | 51.9 | 6.5 | 7.0 |
| 45% | 38.6 | 47.1 | 6.5 | 7.8 |
| 50% | 43.3 | 43.3 | 6.5 | 7.0 |

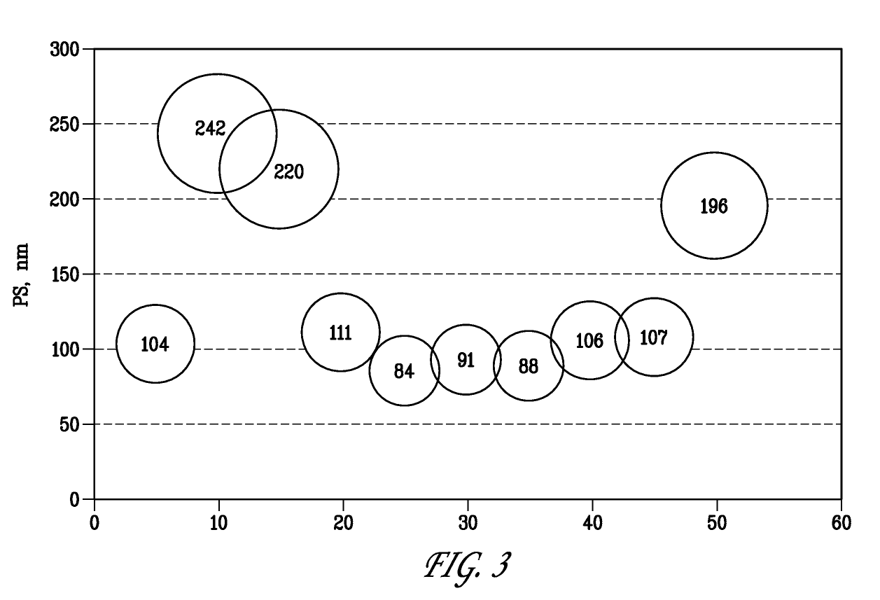

The samples indicated in the first column as 5% to 50% refer to the % surfactant within the vehicle/excipient portion of the formulation only. The % amounts in the remaining columns pertain to the % amounts within total formulation. The results are illustrated in the graph of FIG. 3, showing relative particle size (nm) per varying % amounts of surfactant MYRJ® 52. As can be seen from the figure, particle sizes varied with dilution, and there is a significant reduction in particle size associated with surfactant concentrations ranging between about 20% to about 45% of the total composition weight. Further, it was also observed that different levels of surfactant were stable to dilution.

### Example 9

### Comparative Dissolution with Varying PEG Molecular Weight

To ascertain and evaluate the optimal hydrophilic ingredient—namely PEG molecular weight—the effect of varying PEG molecular weights on dissolution was investigated. In all of the following compositions the lestaurtinib and water concentrations were fixed at 6.6% lestaurtinib and 7.7% water. Two concentrations of MYRJ® 52 in the formulations were tested—one at 5% MYRJ® 52 and a second at 50% MYRJ® 52 amounts. The remainder of the formulation comprised the appropriate PEG. For example, the 5% MYRJ® 52 formulation had 6.6% lestaurtinib, 7.7% water, 5% MYRJ® 52 and 80.7% of the appropriate PEG. The dissolution for each of these base formulations was evaluated using PEG 1000, 1450, 3350, and 4600.

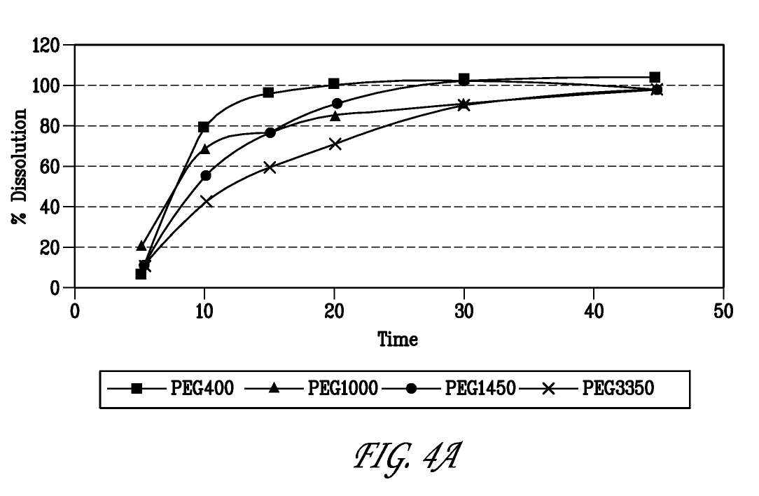

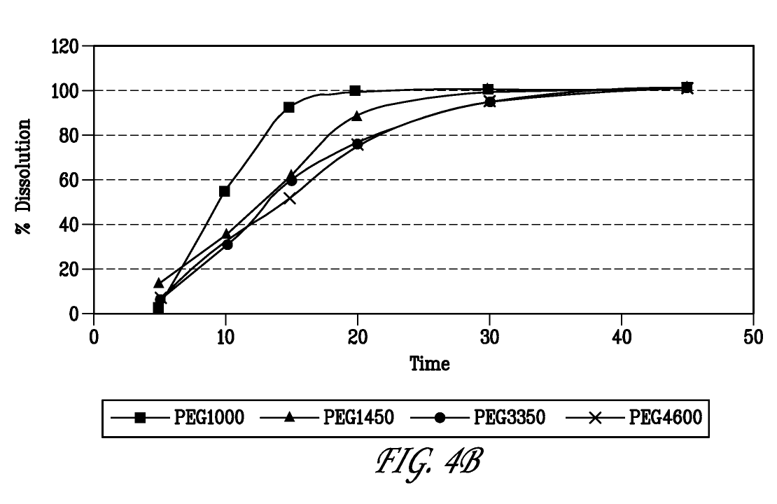

The data for each 5% and 50% amount base formulation was compiled and the results were plotted as percent (%) dissolution versus time as set forth in FIGS. 4A (5% MYRJ® surfactant) and 4B (50% MYRJ® surfactant content). As can be seen from the results, in both the 5% and 50% surfactant concentration base vehicle formulations, there appeared to be little variation in dissolution of the compositions with the various PEG molecular weights ranging from 1000 to 4600. Put another way, PEG molecular weight appears to have little impact on the overall dissolution trend of the active ingredient composition prepared according to the invention.

### Pharmacokinetic Studies—General Procedure

The effects of varying certain ingredients within formulations and associated pharmacokinetic studies were evaluated through several studies using rat models. In each of the studies, formulations were prepared using the following general procedure. The vehicle solution is initially prepared by combining the ingredients, adding the desired amount of lestaurtinib and stirring on a 65° C. hotplate until uniform. While stirring, the appropriate amount of water is added to the slurry. Mixing continued until a clear homogenous solution is obtained. The formulations are then solidified and stored at a temperature of about 5° C. until time of use for the study. The process of preparing the formulations used below varied in terms of the ingredient variable being evaluated for each study.

In each of the studies, adult male Sprague-Dawley rats weighing about 0.3 kg (Charles River, Kingston, N.Y.) were used. Three adult male rats were used in each treatment group unless specified otherwise. The rats were fasted overnight prior to oral dose administration. Prior to administration, the formulations were rewarmed to a temperature of about 45° C. and once liquified, they formulations were orally administered using a positive displacement pipette at a dose of 40 mg/kg body weight.

### Example 10

### Lestaurtinib Formulation PK Studies Using In Vivo Rat Model

The bioavailabilities of various concentrations of lestaurtinib prepared according to the invention were compared to each other as well as to a control formulation. The non-microemulsion control formulation was prepared without the water-addition formed microemulsion of the invention. The remaining formulations contained varying ranges of vehicle stock solution, lestaurtinib, and water. The microemulsion formulation was prepared from a vehicle stock solution that contained a 25% PEG-400/25% PEG-1000/50% MYRJ®52 by weight mixture. The formulation was prepared in 10.0 g quantities by adding the required amount of vehicle to the appropriate amount of lestaurtinib (set forth in Table 6 below). This suspension was slurried on a 65° C. hotplate until uniform. While stirring, the appropriate amount of water was added to the slurry. Mixing continued until a clear homogenous solution was obtained. The formulations were then solidified and stored at a temperature of about 5° C. until time of use for the study.

The study was conducted using adult male Sprague-Dawley rats weighing about 0.3 kg (Charles River, Kingston, N.Y.) housed 3 per cage. Three adult male rats were used in each treatment group. The rats were fasted overnight prior to oral dose administration. Prior to administration, the formulations were rewarmed to a temperature of about 45° C. and once liquified, they formulations were orally administered using a positive displacement pipette at a dose of 40 mg/kg body weight. The formulations tested are set forth in the following table:

TABLE 7
Lestaurtinib Formulations (40 mg/kg)

| Formula: | Control | 1 | 2 | 3 | 4 |
|---|---|---|---|---|---|
| Lestaurtinib | 25.3 | 26.8 | 48.3 | 70.8 | 81.6 |
| | mg/ml | mg/ml | mg/ml | mg/ml | mg/ml |
| | % | | | | |
| Ingredient: | weight | | | | |
| Lestaurtinib | 2.50% | 2.49% | 4.00% | 6.00% | 7.00% |
| Vehicle 25/25/50 (wt %) | | 97.06 | 93.00 | 87.57 | 84.87 |
| PEG-400/PEG-1000/MYRJ52 | | | | | |
| Propylene glycol | 48.75 | — | — | — | — |
| Tween 80 | 48.75 | — | — | — | — |
| Water | — | 0.45% | 3.00% | 6.43% | 8.14% |
| Volume Dosed | 0.48 mls | 0.45 mls | 0.25 mls | 0.17 mls | 0.14 mls |

For blood collection, each rat was placed in a clear plexiglass restraining tube, and blood samples were drawn (approximately 0.25 ml samples) from a lateral tail vein into heparinized collection tubes at sampling times of 0.25, 0.5, 1, 2, 4 and 6 hour intervals. The blood samples were placed on wet ice until centrifuged to separate plasma. The plasma fraction was transferred into clean dry tubes, frozen on dry ice, and stored at a temperature of approximately −20° C. pending LC-MS analysis. The following data was collected from the samples and set forth in the following table.

TABLE 8
Comparative PK Data for Lestaurtinib Oral Dosing (40 mg/kg)

| PK | Control | 1 | 2 | 3 | 4 |
|---|---|---|---|---|---|
| Cₘₐₓ (ng/ml) | 585 | 466 | 585 | 656 | 1147 |
| Tₘₐₓ (hr) | 1.3 | 5.3 | 3.7 | 2.8 | 0.7 |
| AUC₀₋₈ (ng*hr/ml) | 2522 | 1590 | 2204 | 2657 | 4340 |
| AUC_{0-∞} (ng*hr/ml) | ND | ND | ND | ND | 5266 |
| T_{1/2} (hr) | ND | ND | ND | ND | 2.06 |

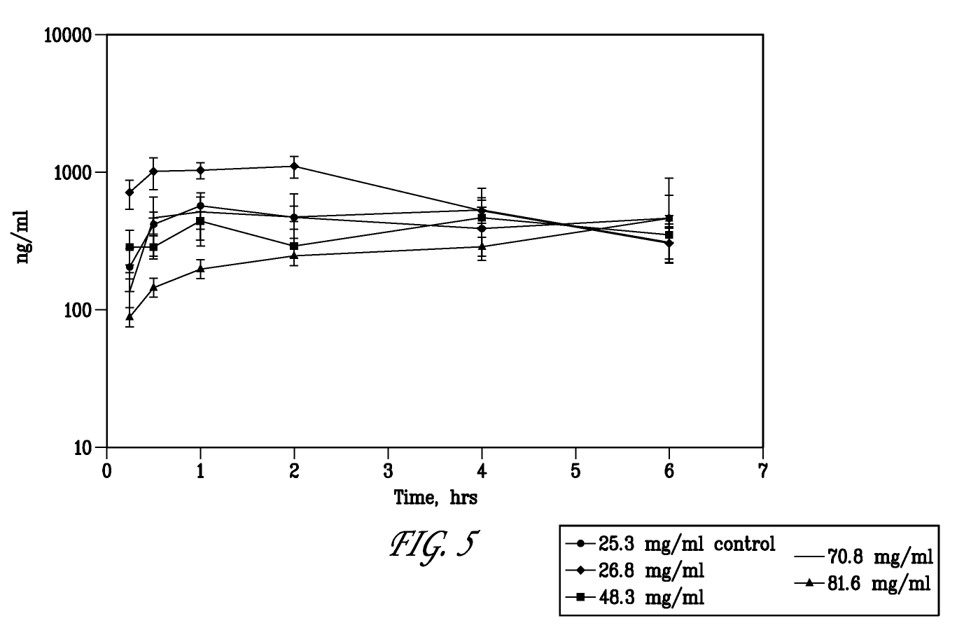

The serum concentrations of active lestaurtinib for the comparative 40 mg/kg lestaurtinib formulations are set forth in the graph of FIG. 5 entitled Plasma Levels of Lestaurtinib in Fasted Rats—Oral Formulation Comparison. Referring now to the figure, it can be seen that serum concentration levels (ng/ml) were comparable over time relative to the non-invention control formulation over time (hrs). Thus, compositions prepared in accordance with the invention at the higher drug concentration permitted substantially increased concentrations of pharmaceutically active lestaurtinib without significant loss or compromise to bioavailability of the active.

### Example 11

### Lestaurtinib Formulation PK Study with Varying Amounts of Surfactant and Hydrophilic Component

Using a process similar to that described above, the following formulations were prepared for the study and administered to the rat models:

TABLE 9
Variable Surfactant % Amount Vehicle Formulation

| | | % PEG-400/PEG-1000 | % | |
|---|---|---|---|---|
| Sample | % MYRJ ® | (50/50 w/w) | Lestaurtinib | % Water |
| 5% | 4.4 | 83.7 | 6.6 | 5.2 |
| 15% | 13.0 | 73.9 | 6.5 | 6.5 |
| 25% | 21.8 | 65.5 | 6.5 | 6.1 |
| 35% | 30.4 | 56.5 | 6.6 | 6.5 |
| 45% | 38.6 | 47.1 | 6.5 | 7.8 |
| 50% | 43.3 | 43.3 | 6.5 | 7.0 |

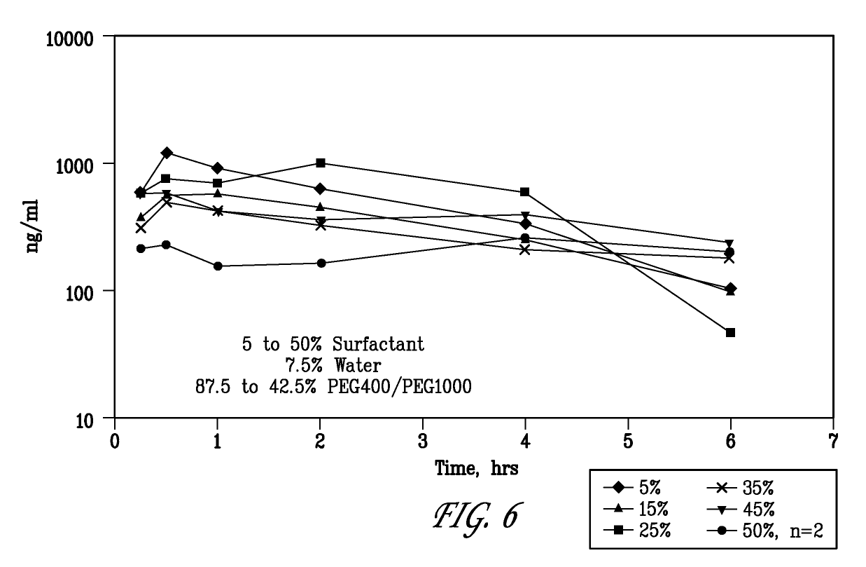

Thus, the formulations were prepared in accordance with a process similar to that described in the general procedure above, and were prepared on the basis of varying the amount of surfactant within the vehicle per se ranging from 5% to 50% alongside an adjusted total amount of hydrophilic component (PEG combination of consistent formula PEG-400/PEG-1000). The formulations were administered using the rat model and procedure described above in the general procedure, and serum samples were collected and analyzed. The resulting data was plotted and appears in the graph of FIG. 6 (the lower left key indicating % amounts within the vehicle/excipient portion of the formulation per se).

As can be seen from the data, some variations of surfactant concentration are possible without significantly adversely affecting the bioavailability of lestaurtinib when prepared according to the invention.

### Example 12

### Lestaurtinib Formulation PK Study with Varying PEG Molecular Weights

Using a process similar to that described above, the following formulations were prepared for the study and administered to the rat models:

TABLE 10
Varying PEG Molecular Weight Formulations

| Ingredient: | lestaurtinib | water | MYRJ | PEG |
|---|---|---|---|---|
| Sample 1 | 6.6% | 7.5% | 5.0% | 80.9% PEG-400 |
| Sample 2 | 6.6% | 7.5% | 5.0% | 80.9% PEG-1000 |
| Sample 3 | 6.6% | 7.5% | 5.0% | 80.9% PEG-1450 |
| Sample 4 | 6.6% | 7.5% | 5.0% | 80.9% PEG-3350 |
| Sample 5 | 6.6% | 7.5% | 5.0% | 80.9% PEG-4600 |

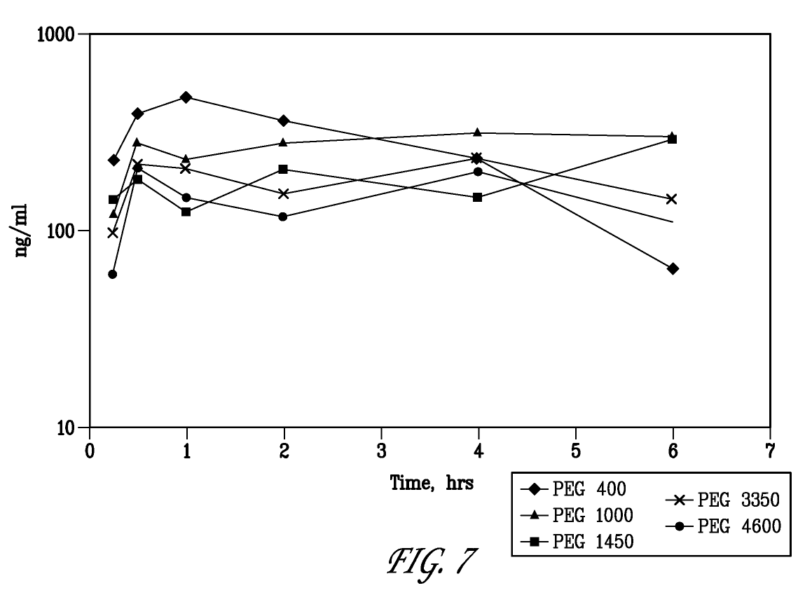

Thus, the above five formulations contained a consistent overall composition relative to the type of ingredients and while maintaining proportions thereof, varied within the hydrophilic component of 80.9% the molecular weight species of polyethylene glycol (i.e., PEG-400, 1000, 1450, 3350 and 4600). The formulations were prepared using the general procedure described above (i.e., stirring molten PEG and MYRJ® in a beaker on 65° C. hotplate, adding lestaurtinib and stirring for about 5 minutes and adding water until clear solution obtained). The formulations were then administered using the rat model as described in accordance with the general procedure as well. The serum samples were collected and analyzed for lestaurtinib content, and the data was calculated and plotted in the graph of FIG. 7.

As can be seen from the data, variations in the molecular weight of PEG as the hydrophilic component within formulations prepared according to the invention appear to have little impact on the bioavailability of lestaurtinib in the rat models. Thus, a range of PEG can be used as the hydrophilic component in formulations prepared according to the invention.

### Example 13

### Preparation of Lestaurtinib Microemulsion Composition without Surfactant

In one embodiment, microemulsions containing solubilized indolocarbazole compounds, e.g., lestaurtinib, can be prepared using the hydrophilic component and water in the excipient composition but without the presence of the surfactant, e.g., MYRJ®. In one example, a composition without surfactant was prepared as follows. Lestaurtinib 120 mg was weighed into a glass vial and 2 mL of molten PEG-1450 was added. The slurry was then heated to approximately 55° C. and stirred for about 1 hour at which time the mixture appeared to be a slurry or suspension of Lestaurtinib and PEG-1450. Next, 200 μL of water was added and the solution and the mixture was stirred an additional 20 minutes resulting in complete solubilization of the lestaurtinib and a clear solution. The final concentration of lestaurtinib in this resulting clear solution was 59.2 mg/mL as determined by high pressure liquid chromatography. Thus, it is possible to prepare highly solubilized concentrations of indolocarbazole compounds in microemulsion form without the use of a surfactant.

### Example 14

### Preparation of a 2 kg Batch of Tablet Blend for Compression into 20 mg Dose Lestaurtinib Tablets Having a Target Weight of 600 mg

In another embodiment, microemulsions containing solubilized indolocarbazaole compounds, e.g., lestaurtinib, can be combined with commonly used excipients to prepare tablet dosage forms.

A blend suitable for compression of 20 mg lestaurtinib tablets can be produced by first formulating the microemulsion, absorbing the microemulsion formulation into magnesium aluminometasilicate, and then mixing the loaded magnesium aluminometasilicate with binder, disintegrant and lubricant. A 20 mg dose can then be manufactured by compressing the blend with a target tablet weight of 600 mg.

To prepare a 2 kg batch of blend, the microemulsion is formulated by blending 20 grams of melted PEG-1000 with 20 grams of melted MYRJ-52. The mixture is heated and stirred at approximately 55° C. until homogenous. 3.6 grams of lestaurtinib is weighed into a suitable container and 36.9 grams of the PEG-1000/MYRJ 52 blend is added. This mixture is heated at approximately 55° C. for 20 minutes. 4.5 grams of water is then added and the mixture is stirred until a clear microemulsion results.

15 grams of magnesium aluminometasilicate is weighed into a container and heated to approximately 60° C. The entire 45 grams of lestaurtinib microemulsion is then slowly added with constant stirring to the magnesium aluminometasilicate. The mixture is spread on foil to cool.

55.5 grams of the lestaurtinib and magnesium aluminometasilicate mixture is weighed into a 250 cc amber glass bottle. 32.5 grams microcrystalline cellulose, 5 grams lactose, and 5 grams sodium starch glycolate are added and the mixture is blended in a turbula mixer for 6 minutes. 2 grams of magnesium stearate is then added and the mixture is blended for an additional three minutes. The blend is then removed and compressed into 20 mg lestaurtinib tablets with a tablet weight of 600 mg.

TABLE 11
Formulation for 20 mg Lestaurtinib Tablets

| | | Tablet amount | |
|---|---|---|---|
| Component | Final % | (mg) | Batch quantity (g) |
| lestaurtinib | 3.333 | 19.998 | 66.660 |
| Water | 4.163 | 24.978 | 83.260 |
| PEG-1000 | 17.070 | 102.397 | 341.400 |
| MYRJ-52 | 17.070 | 102.397 | 341.400 |
| Magnesium | 13.875 | 83.220 | 277.500 |
| aluminometasilicate | | | |
| Microcrystalline | 32.500 | 195.000 | 650.000 |
| cellulose | | | |
| Lactose | 5.000 | 30.000 | 100.000 |
| Sodium starch glycolate | 5.000 | 30.000 | 100.000 |
| Magnesium stearate | 2.000 | 12.000 | 10.000 |
| Total | 100 | 600 | 2000 |

### Pharmaceutical Methods

The invention includes a method of inhibiting receptor-tyrosine kinase in a recipient comprising administering to the recipient a therapeutically effective amount of indolocarbazole compound in a dosage form, the dosage form comprising the pharmaceutical composition of the invention described herein above. Based on the in vivo rat studies above, the active ingredient lestaurtinib can be successfully delivered in serum concentrations needed for successful therapeutic effect in the mammalian model. It follows then that, upon administering the oral dosage form containing the indolocarbazole compound lestaurtinib as prepared in accordance with the invention, the effective therapeutic biomechanism known to be associated with lestaurtinib, i.e., inhibition of tyrosine receptor kinase, occurs.

Thus, various treatments and therapies using lestaurtinib and other pharmaceutically active indolocarbazole compounds can be effected by administering oral dosage forms of the invention—particularly the encapsulated microemulsion compositions prepared according to the invention. Indolocarbazole compounds such as lestaurtinib are known to be useful in the treatment of various diseases and disorders. Therefore, it is expected that the composition and dosage form of the invention as administered to an individual in need of such a treatment associated with lestaurtinib is possible.

### INDUSTRIAL APPLICABILITY

The invention is useful in the preparation of dosage forms containing liquid fill compositions having one or more indolocarbazole compounds as the active ingredient. The invention is useful for the preparation of capsular dosage forms having increased concentrations of the active ingredient, thereby reducing capsule size and/or daily administration episodes.

The invention herein above has been described with reference to various publications, e.g., patents and patent applications. The full text of each reference is incorporated herein by reference.

The invention has been described herein above with reference to various and specific embodiments and techniques. It will be understood by one of ordinary skill, however, that reasonable modifications and variations may be made of such embodiments and techniques without substantially departing from either the spirit or scope of the invention as defined by the following claims.

What is claimed is:

1. A pharmaceutical composition comprising:
an indolocarbazole compound present in a solubilized concentration ranging from about 3% up to about 9% by weight of the total composition; a hydrophilic polymer component; and water; wherein said composition is a microemulsion.

2. The composition according to claim 1, wherein said indolocarbazole is lestaurtinib or [9S-(9α, 10β,12α)]-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one.

3. The composition according to claim 1, wherein said indolocarbazole compound is lestaurtinib and said lestaurtinib is present in a solubilized concentration of between about 3% and about 9% of the total composition weight of the microemulsion.

4. The composition according to claim 1, wherein said hydrophilic polymer component comprises polyethylene glycol.

5. The composition according to claim 1, wherein said composition further comprises a surfactant.

6. The composition according to claim 5, wherein said surfactant is selected from the group consisting of anionic surfactants, cationic surfactants, and nonionic surfactants, and combinations thereof.

7. The composition according to claim 6, wherein said surfactant is a polyoxyethyl stearate.

8. The composition according to claim 1, further comprising an antioxidant component selected from the group consisting of H-atom donor antioxidant, sacrificial antioxidants and oxygen scavenger antioxidants, and combinations thereof.

9. The composition according to claim 8, wherein said antioxidant is selected from the group consisting of vitamin E, ascorbic acid, KMBS (potassium metabisulfite salt), ascorbyl palmitate, sodium ascorbate, BHA (butylated hydroxyanisole), BHT (butlyated hydroxytoluene) and combinations thereof.

10. An oral capsular dosage form comprising a pharmaceutical fill composition encapsulated within a capsule material, said composition comprising:
a) indolocarbazole compound present in an amount ranging from about 3% to about 9% per total composition weight;
b) a hydrophilic polymer component present in an amount ranging from about 30% to about 95% per total composition weight; and
c) water present in an amount ranging from about 0.8% to about 50% by weight of total composition weight;
wherein said composition is a microemulsion formulated for encapsulation with a hard capsule material.

11. The dosage form according to claim 10, wherein said capsule material is a hard capsule material selected from the group consisting of gelatin and hydroxypropylmethylcellulose.

12. The dosage form according to claim 10, wherein said indolocarbazole compound is lestaurtinib.

13. The dosage form according to claim 12, wherein said composition comprises:
a) lestaurtinib present in an amount from about 3% to about 9% total composition weight;
b) hydrophilic polymer component comprising a polyethylene glycol present in an amount from about 30% to about 90% total composition weight;
c) a surfactant comprising a polyoxyl stearate and present in an amount from about 5% to about 45% total composition weight;
d) water present in an amount from about 0.8% to about 15% total composition weight; and
e) an antioxidant component comprising a mixture of antioxidants and present in an amount from about 0.1% to about 2% total composition weight;
wherein said composition is in the form of a microemulsion and encapsulated within a hard capsule.

14. The dosage form according to claim 13, where said composition comprises:
a) lestaurtinib present in an amount from about 5% to about 7% total composition weight;
b) PEG-1000 present in an amount from about 35% to about 45% total composition weight;
c) MYRJ® 52 present in an amount from about 35% to about 45% total composition weight;
d) water present in an amount from about 6% to about 8% total composition weight;
e) vitamin E present in an amount which is about 0.075% total composition weight;
f) ascorbyl palmitate present in an amount which is about 0.1% total composition weight;
g) ascorbic acid present in an amount which is about 0.1% total composition weight; and
h) KMBS present in an amount which is about 0.2% total composition weight;
wherein said composition is in the form of a microemulsion and encapsulated within a hard capsule.

15. The dosage form according to claim 13, where said composition comprises:
a) lestaurtinib present in an amount from about 5% to about 7% total composition weight;
b) PEG-1000 present in an amount from about 35% to about 45% total composition weight;
c) MYRJ® 52 present in an amount from about 35% to about 45% total composition weight;
d) water present in an amount from about 6% to about 8% total composition weight;
e) vitamin E present in an amount which is about 0.075% total composition weight;
f) ascorbyl palmitate present in an amount which is about 0.1% total composition weight;
g) sodium ascorbate present in an amount which is about 0.125% total composition weight; and
h) KMBS present in an amount which is about 0.2% total composition weight;
wherein said composition is in the form of a microemulsion and encapsulated within a hard capsule.

16. The dosage form according to claim 13, where said composition comprises:
a) lestaurtinib present in an amount from about 5% to about 7% total composition weight;
b) PEG-1000 present in an amount from about 35% to about 45% total composition weight;
c) MYRJ® 52 present in an amount from about 35% to about 45% total composition weight;
d) water present in an amount from about 6% to about 8% total composition weight;
e) vitamin E present in an amount which is about 0.15% total composition weight;
f) ascorbyl palmitate present in an amount which is about 0.2% total composition weight; and
g) ascorbic acid present in an amount which is about 0.05% total composition weight;
wherein said composition is in the form of a microemulsion and encapsulated within a hard capsule.

17. The dosage form according to claim 13, where said composition comprises:
a) lestaurtinib present in an amount from about 5% to about 7% total composition weight;
b) PEG-1000 present in an amount from about 35% to about 45% total composition weight;
c) MYRJ® 52 present in an amount from about 35% to about 45% total composition weight;
d) water present in an amount from about 6% to about 8% total composition weight;
e) vitamin E present in an amount which is about 0.15% total composition weight; and
f) ascorbyl palmitate present in an amount which is about 0.2% total composition weight;
wherein said composition is in the form of a microemulsion and encapsulated within a hard capsule.

18. A pharmaceutical composition comprising a microemulsion having an indolocarbazole compound as an active ingredient present in a solubilized concentration ranging from about 3% up to about 9% of the total composition weight, said composition being prepared by:
a) combining an indolocarbazole compound, hydrophilic polymer component, and surfactant;
b) adding water to the combined ingredients from step a) in an amount sufficient to increase the solubilized concentration of said indolocarbazole compound to the desired concentration amount;
wherein steps a) and b) are performed at a temperature sufficient to form a molten liquid of the combined ingredients, facilitate solubilization of said indolocarbazole compound and form a microemulsion.

19. A process for increasing the solubilized concentration of an indolocarbazole compound in a microemulsion for a given fill volume, said process comprising:
a) combining an indolocarbazole compound, hydrophilic polymer component, and surfactant;
b) adding water to the combined ingredients from step a) in an amount sufficient to increase the solubilized concentration of said indolocarbazole compound to the desired concentration amount;
wherein steps a) and b) are performed at a temperature sufficient to form a molten liquid of the combined ingredients, facilitate solubilization of said indolocarbazole compound and form a microemulsion;
wherein said indolocarbazole is present in an amount of up to about 9% total composition weight.

20. A method of inhibiting receptor-tyrosine kinase in a recipient comprising orally administering to a recipient in need of such treatment an oral dosage form having a pharmaceutical composition comprising:
a) lestaurtinib present in a solubilized concentration of from about 3% to about 9% by weight of the total composition;
b) a hydrophilic polymer component comprising polyethylene glycol;
c) a surfactant; and
d) water;
wherein said composition is a microemulsion.

21. An oral tablet dosage form comprising a pharmaceutical composition, said composition comprising:
a) indolocarbazole compound present in an amount ranging from about 3% to about 9% per total composition weight;
b) a hydrophilic polymer component;
c) water; and
d) additional pharmaceutically acceptable excipients;
wherein said composition is a microemulsion formulated for compression into a tablet dosage form.

22. The dosage form according to claim 21, wherein said composition comprises:
a) lestaurtinib present in an amount which is about 3% total composition weight;
b) PEG-1000 present in an amount which is about 17% total composition weight;
c) water present in an amount which is about 4% total composition weight; and
d) additional pharmaceutically acceptable excipients selected from the group consisting of magnesium aluminometasilicate, microcrystalline cellulose, lactose, sodium starch glycolate and magnesium stearate;
wherein said composition is a microemulsion formulated for compression into a tablet dosage form.
